(19) United States
(12) Patent Application Publication
(10) Pub. No.: US 2008/0281204 A1
(43) Pub. Date: Nov. 13, 2008

(54) System and Method for Treating Hemorrhoids

(76) Inventors: Raffaele Salfi, Cagliari (IT); Wolfgang Neuberger, Labuan (MY)

Correspondence Address: MCCARTER & ENGLISH LLP;CITYPLACE I, 185 ASYLUM STREET, HARTFORD, CT, 06103, US

(21) Appl. No.: 12/119,091

(22) Filed: May 12, 2008

Related U.S. Application Data
(60) Provisional application No. 60/928,719, filed on May 11, 2007.

(51) Int. Cl. A61B 8/00; A61F 7/00

(52) U.S. Cl. 600/454; 600/453; 607/98

(57) ABSTRACT

A system and method are presented for treating branches of the superior hemorrhoidal artery by photocoagulation using laser energy. The inflamed dilated blood vessels in and around the anal region, often called hemorrhoids or piles, are caused due to a connective tissue disorder, a relative increase in pressure in the superior hemorrhoidal artery and a weakening of the vessels' valves. A suitable treatment system and a method are given for treating such conditions in a minimally invasive manner. The treatment system photocoagulates the branches of a superior hemorrhoidal artery in the anal and rectal regions using laser energy while causing minimal pain or discomfort to the patient. The system is provided with a transparent operational window. The distal end of the fiber and Doppler system channel are at the operational window. The system also includes a viewing window. The post operative recovery is faster than alternative approaches with no complications. In contrast to most other successful methods of the prior art, general anesthesia is no longer required, thereby significantly reducing complications and simplifying treatment.

This application claims the benefit of U.S. Provisional Application Ser. No. 60/928,719, filed May 11, 2007, which is incorporated by reference herein.

### FIELD OF THE INVENTION

The present invention relates to a system and method for treating hemorrhoids in patients using a minimally invasive technique, and more particularly, to a method and a treatment system using a monitoring device, such as a Doppler or ultrasound device, and an energy application device, such as a fiber optic or other intensive radiation device, to thermally damage hemorrhoidal arteries to reduce or prevent blood flow therein.

### BACKGROUND OF THE INVENTION

Hemorrhoids are dilated or bulging veins of the rectum and anus, caused by increased pressure in the rectal veins, resulting in bleeding, itching and pain. Pressure on the walls of the rectum weakens the muscles that support the hemorrhoidal vessels. The vessels in turn become enlarged and lose their support forming a sac-like protrusion inside the rectal canal, referred to as internal hemorrhoids, or under the skin around the anus, referred to as external hemorrhoids. When internal hemorrhoids push out of the anal opening, it is referred to as a prolapsed hemorrhoid. Sometimes, a blood clot or thrombus can be seen in external hemorrhoids, and this condition is referred to as a thrombosed external hemorrhoid. Hemorrhoids can occur at any time, in all age groups, and in both sexes. Younger people, pregnant women and women who have had children are also reported to encounter hemorrhoidal problems.

The arteries supplying blood to the anal canal descend into the canal from the rectum above, and form a rich network of arteries that communicate with each other around the anal canal. Because of this rich network of arteries, hemorrhoidal blood vessels have a ready supply of arterial blood. The blood vessels that supply the hemorrhoidal vessels pass through the supporting tissue of the hemorrhoidal cushions. The anal veins drain blood away from the anal canal and the hemorrhoids. These veins drain in two directions. The first direction is upward into the rectum, and the second is downward beneath the skin surrounding the anus. The suggestion based on earlier theories of hemorrhoid aetiology is that a local increase in venous pressure causes dilation of the hemorrhoid plexus within the anal cushions. However, this theory has been refuted based on recent studies of the vascular anatomy of the anal cushion.

The common symptoms of hemorrhoids are bleeding and prolapse at defecation. Thromboses and anal fissures associated with hemorrhoids can cause pain. Other symptoms of hemorrhoids include soiling, itching and perianal irritations.

For moderate hemorrhoid conditions, there are numerous existing treatments, the main objectives of which are to alleviate the symptoms or to help avoid aggravation of the hemorrhoid condition. Such treatments include ointments, special diets, patches and hemorrhoid massage, which is used to stimulate blood flow in the treatment area.

Another general treatment method is anal dilation. Anal dilation is a conventional method wherein the anal sphincter muscle is stretched or dilated to prevent hemorrhoids from increasing rectal pressure, as well as to reduce straining while passing stool. Potential side effects of this procedure are fecal incontinence or anal leakage. Accordingly, this method cannot be used in older age persons and/or persons with weak sphincter muscles.

In rubber-band ligation methods a special rubber band is tied around internal hemorrhoids to cut off the circulation within the hemorrhoid. The band cuts off blood circulation to the hemorrhoid, which in turn shrivels and falls off with the band in a about week's time. In the case of multiple hemorrhoids, they are treated separately at intervals of about one month apart. Common side effects of rubber-band ligation include complications such as clotting of external hemorrhoids and bleeding.

In Doppler-guided ligation methods a specially adapted protoscope with a Doppler probe incorporated therein is used to ligate hemorrhoid arteries. The Doppler probe is inserted and used to locate the hemorrhoid arteries by audible alteration of the Doppler signal. Once located, a needle holder is inserted into the lumen of the protoscope and the artery is ligated with an absorbable suture into the submucosa. The procedure is repeated. Doppler-guided ligation of the hemorrhoid artery disrupts the arterial inflow and tethers the mucosa, causing the hemorrhoidal mass to shrink and retract. Currently this procedure is carried out under general anesthesia and few patients are able to tolerate it under simple sedation.

Sclerotherapy or injection therapy involves injecting a sclerosing or hardening agent into the base of internal hemorrhoids. The sclerosing agent causes the vein walls to collapse and the hemorrhoids to shrivel up. This method can be used for treating multiple hemorrhoids at once and is more often used for treating older men and women with fragile veins. One of the drawbacks associated with this method is that abscesses have been reported in some patients.

Cryosurgery or freezing methods use liquid nitrogen or nitrous oxide to cool the cyroprobe to a freezing temperature. The frozen tip of the probe is then moved into contact with the hemorrhoid to freeze and destroy the tissue. Two or three weeks later, the hemorrhoid shrinks and falls off. Both internal and external hemorrhoids can be treated by this method. One of the drawbacks of this method is that it can be very painful and other complications can arise, such as the discharge of foul odors from the treated hemorrhoids that can last for about a week or so and require the use of absorbent pads. Another drawback associated with this method is that the wound can become infected.

Hemorrhoidectomy is a surgical method of removing hemorrhoids and is usually employed in severe cases. This method is recommended for prolapsed or thrombosed internal hemorrhoids, or large and painful external hemorrhoids. In this method, hemorrhoids are surgically removed using scalpels or lasers, the cut is sewn with stitches, and a small pad is placed in the anus to absorb discharge from the treated region. This method requires anesthesia and hospitalization for a few days followed by bed rest. This method therefore is relatively expensive. Side effects of this method can include severe pain, bleeding, narrowing of the anal canal which can, in turn, lead to anal fissures, an inability to defecate, and scarring.

Electric treatments of hemorrhoids apply an electric current directly into the deficient vein. The current, negative or positive, causes a chemical or thermal reaction within the tissue that either destroys and/or obliterates the hemorrhoid. Examples of these treatments include bipolar electrotherapy and hemorrhoidolysis, in which therapeutic galvanic waves are applied directly to the hemorrhoid, producing a chemical reaction that shrinks and dissolves hemorrhoidal tissue. These treatments are limited to internal hemorrhoids. Furthermore, they can be time-consuming treatments, tedious for physicians and patients, and can lead to the development of anorectal fistulas.

Recent infrared radiation methods have used the heat generated by an infrared device to destroy hemorrhoids. The device is used to coagulate the hemorrhoid and in turn shrink it. This method is an improvement over earlier methods because it allows for improved control over the depth of coagulation. While post-operative complications can be minimized and less bleeding has been reported, this procedure still has problems associated with placement and application of the radiation and pain.

Hence, there remains a need for developing a suitable device and method for treating hemorrhoids using minimally invasive methods which can overcome and/or minimize one or more of the above-described drawbacks and/or disadvantages of the prior art.

### OBJECTIVES AND BRIEF SUMMARY OF THE INVENTION

It is an objective of the present invention to provide a treatment system for treating hemorrhoids in patients.

It is another objective of the currently preferred embodiments of the present invention to provide a laser treatment system for treating hemorrhoids in patients.

It is a further objective of the currently preferred embodiments of the present invention to provide a method of treating hemorrhoids by photocoagulating hemorrhoid vessels.

Briefly stated the present invention is a system and method for treating branches of the superior hemorrhoidal artery by applying thermal energy, such as by photocoagulation using laser energy. The inflamed dilated blood vessels in and around the anal region, often called hemorrhoids or piles, are caused due to a connective tissue disorder, a relative increase in pressure in the superior hemorrhoidal artery and a weakening of the vessel valves. A suitable treatment system and a method are provided herein for treating such conditions in a minimally invasive manner. In the currently preferred embodiments of the present invention, the treatment system photocoagulates the dilated blood arteries in the anal and rectal regions using laser energy while preferably causing minimal pain or discomfort to the patient. The treatment system is provided with a an anus-scope defining a size and/or shape for passage into and out of the anal canal and defining one or more channels therethrough, a monitoring device, such as a Doppler or ultrasound probe, that is receivable within the channel for locating the hemorrhoidal artery to be treated, and an energy application device, such as a fiber optic line coupled to a laser, that is receivable through the channel for applying energy, such as laser energy, to the identified hemorrhoidal artery to thermally damage the artery and, in turn, significantly reduce and/or terminate blood flow to the hemorrhoid. One advantage of the system and method of the present invention is that it can allow for more rapid post operative recovery in comparison to prior art treatments and methods preferably with fewer and/or no complications. Yet another advantage of the system and method of the present invention is that general anesthesia is not required, as it is in most other successful methods, greatly reducing complications and simplifying treatment.

The above and other objects, features and advantages of the present invention and/or of the currently preferred embodiments thereof will become apparent from the following description read in conjunction with the accompanying drawings.

### BRIEF DESCRIPTION OF THE DRAWINGS

FIG. 1 is a schematic illustration of a first embodiment of a treatment system of the present invention having a disposable or reusable anus-scope and fiber optics;

FIG. 2A is an end elevational view of a second embodiment of an anus-scope of the present invention; and

FIG. 2B is a side, partial cross-sectional view of the anus-scope of FIG. 2A.

### DETAILED DESCRIPTION OF PREFERRED EMBODIMENTS

Hemorrhoids, also commonly referred to as piles, when visible, are conditions found to occur in affected veins in and around the anal or rectal areas. It is a condition found to affect about 30% of the general population.

The arteries supplying blood to the anal canal descend into the canal from the rectum above and form a rich network of arteries that communicate with each other around the anal canal. Because of this rich network of arteries, hemorrhoidal blood vessels have a ready supply of arterial blood. The blood arteries that supply the hemorrhoidal vessels pass through the supporting tissue of the hemorrhoidal cushions. The anal veins drain blood away from the anal canal and the hemorrhoids. These veins drain in two directions. The first direction is upward into the rectum, and the second is downward beneath the skin surrounding the anus. The suggestion based on earlier theories for hemorrhoid aetiology is that a local increase in venous pressure causes dilation of the hemorrhoid plexus within the anal cushions. However, this theory has been refuted based on recent studies of the vascular anatomy of the anal cushion.

Despite their vascular appearance and tendency to bleed, the development of hemorrhoids may be due to a connective tissue disorder and an absolute or relative increased blood flow from the superior hemorrhoidal artery. Arteriovenous anastomoses within the submucosa are thought to contribute to the increase in volume of the anal cushions. This arterial component explains why hemorrhoidal bleeding has the appearance and pH of arterial blood. Based on these new theories, new treatment methods are proposed.

The currently preferred embodiments of the present invention provide a new method and system for treating hemorrhoid conditions based on recently developed theories of hemorrhoidal aetiology. In the currently preferred embodiments, laser energy is used to photocoagulate the arteries supplying the hemorrhoids, without the use of general anesthesia and, as a result, less post operative care is required than in many prior art methods and systems.

The treatment device is provided with a disposable or reusable anus-scope and component. The appropriately sized anus-scope is internally provided with one or more channels for introducing the component. In the currently preferred embodiments, the component of the treatment system includes an energy application device, such as a laser source and fiber optic line coupled thereto, a monitoring device, such as a Doppler device (Doppler, echo-Doppler, and echo-color-Doppler) or ultrasound device, an aspirating device, such as an aspirator for aspirating fumes and/or heat, and a light source for illuminating the scope and/or field. In some such embodiments, a Doppler transducer is used to identify and position the distal end of the scope to treat the branching hemorrhoidal arteries. In some embodiments, the system includes a multi-use channel for receiving therein multiple components, such as an optical fiber, Doppler system, and connection to an aspirating device to exhaust fumes, heat, fluids and/or blood, as well as a channel for a light source for illuminating the scope and/or field. The system also preferably includes a viewing window.

The method of treating the branches of hemorrhoidal superior arteries using the treatment system in general involves inserting the anus-scope into the anal canal above the hemorrhoidal cushions and irradiating the treatment area with laser energy. The branches of hemorrhoidal arteries are located by a Doppler transducer or other methods known in the art. After locating the vessels, laser energy is applied to the artery branches using an optical fiber having a size within the range of about 200-2000 microns. The fiber is introduced into and/or around the artery by viewing with a Doppler system to a depth of about 5-7 cm from the skin line. Emission of laser irradiation from the emitting face or otherwise from the output end of the fiber optic line leads to photocoagulation of the respective hemorrhoidal artery branches. The time and laser power required for complete closure of the hemorrhoidal artery branches are monitored under Doppler or ultrasound control. The complete closure is confirmed by Doppler. One advantage of this system and method is that they provide a minimally invasive out-patient procedure with no requirement for general anesthesia.

The photocoagulation of terminal branches of hemorrhoidal arteries with the laser system of the present invention has many advantages when compared to other treatment methods. Use of the appropriately sized anus-scope is relatively painless and causes less discomfort to the patient when inserted into the anal opening. Since general anesthesia is not used, the patient can be discharged shortly after treatment. The procedure is easy to do and can be performed in a few minutes with minimal or no post-operative pain. Further, with no anesthesia required, there are no related complications.

During the procedure, the position of the optical fiber near the hemorrhoidal cushion can be monitored using the monitoring devices, such as a Doppler or ultrasound device. This also prevents damaging of other normal vessels in the rectal and anal regions. Complete closure of each damaged artery is confirmed by the Doppler system. The sclerosis of the submucosa with fixation of the planes of the rectal wall prevents prolapse. The patient can return to normal activity after the procedure with minimal or no post operative pain.

The present invention is further illustrated by the following examples, but is not limited thereby.

A Treatment System for Treating Hemorrhoidal Cushions:

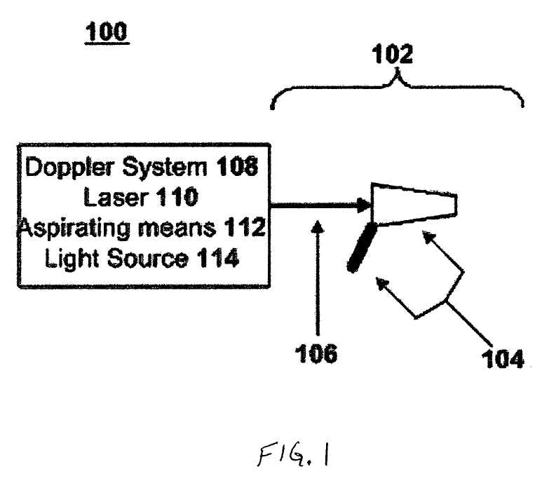

As shown in FIG. 1, the treatment system 100 includes a disposable or reusable hand-piece 102, and the hand-piece 102 includes an anus-scope 104 and fiber optic 106 used for treating the relevant vessel in the anal region. The anus-scope 104 is inserted into the anal canal of the patient with minimal discomfort and without general anesthesia. A Doppler system 108 assists in the location and position of the artery branches that need to be treated by photocoagulation. The located arterial branches are irradiated by laser radiation from a laser 110 optically coupled to the fiber optic 106 for a preset time interval, and the complete closure of the vessel is monitored by the Doppler 108. Fumes, and if necessary, heat are removed during the procedure using an aspiration device 112, such as a smoke evacuator of a type known to those of ordinary skill in the pertinent art. A light source 114 is optically coupled to the anus-scope 104 to illuminate the interior of the anus-scope and/or to illuminate the treatment field by transmitting visible light through a channel of the scope. Partial and/or complete closure of each arterial branch supplying blood to the hemorrhoidal cushion is accomplished by photocoagulation and is confirmed by the Doppler image. After the procedure the anus-scope is removed and the patient can be discharged.

### Example 1

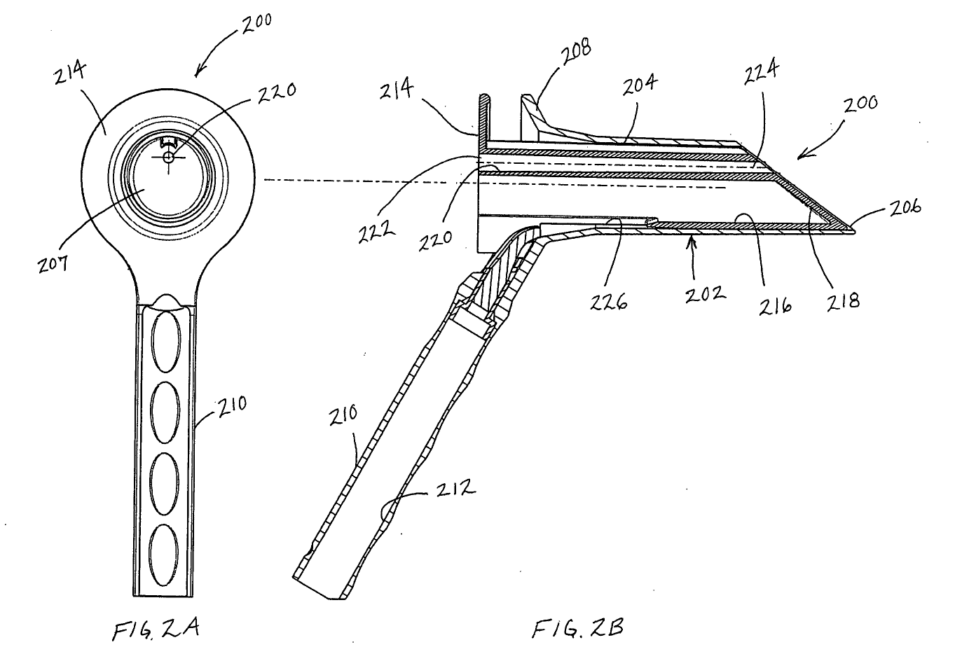

FIGS. 2A and 2B illustrate a disposable or reusable anus-scope 200 designed for easy passage into and out of the anal canal. The anus-scope 200 includes a scope body 202 defining a substantially cylindrical outer wall 204, an angled tip 206 formed on its distal end, and a radially extending flange 208 formed on its proximal end. As can be seen, the angled tip 206 is configured to facilitate ease of insertion of the scope into the anal canal. A handle 210 projects laterally from the proximal end of the body 202, and defines therein a channel 212 that is coupled to a light source for transmitting light therethrough to illuminate the interior of the scope body 202 and/or the treatment field at the distal tip 206 of the scope. An insert 214 is received within the scope body 202 and defines a side wall 216 that is slidably received within the cylindrical wall 204 of the scope body, and an angled distal tip wall 218 that is aligned and flush with the distal tip 206 of the scope body. The insert 214 further defines a multi-use channel 220 extending therethrough and defining an inlet end 222 for receiving therein a plurality of different components, and an outlet end 224 for receiving therethrough the working end or other portion as required of a respective component. In the currently preferred embodiments, the channel 220 is provided: (1) for introduction of the monitoring device, such as the Doppler or ultrasound probe, for detection of the hemorrhoid cushion; (2) for introduction therethrough of the energy application device, such as the optical fiber, for applying energy to a respective hemorrhoidal artery to thermally damage the artery and, in turn, reduce or terminate the flow of blood to the respective hemorrhoid; and (3) for connection to an aspirating devices, such as an aspirator for removing fumes and/or heat. The channel 212 is provided for introducing therethrough an illumination system for illuminating the inside of the anus-scope for viewing and/or the treatment field. If desired, the scope body 202 and insert 214 may be substantially transparent or translucent to facilitate viewing therethrough. The scope 200 defines on its proximal end a viewing opening or window 207, and defines in the side walls 204 and 216 thereof a substantially transparent or translucent operating window 226 for viewing therethrough during a procedure.

### Example 2

### Method of Treating Hemorrhoid Cushions

The anus-scope 200 of FIGS. 2A and 2B is introduced into the anal canal above the hemorrhoidal cushion. The branches of hemorrhoidal arteries are detected by the ultrasound Doppler CW or echo-color-Doppler introduced into the body through the channel 220. The channel 220 is also provided for delivery of the optical fiber which, in turn, delivers laser radiation to the hemorrhoid artery. The illumination system introduced into channel 212 provides light for viewing and illuminating the anus-scope internally. The system is provided with a viewing window 207. Laser energy is applied through an optic fiber defining a size within the range of about 200-2000 microns for a preset time interval required for closing the blood vessel. The complete closure of the blood vessel is verified by using Doppler. A diode laser source is used for photocoagulation of the vessels. After completion of the procedure, the anus-scope is extracted.

### Example 3

### Method of Treating Hemorrhoid Cushions

An anus-scope of the type shown in FIGS. 2A and 2B is provided with an energy source, such as a laser (e.g., a diode laser, such as a 980 nm diode laser), an energy application device, such as a fiber optic line connected to the laser, and receivable through the channel 220, a monitoring device, such as a Doppler probe, receivable within the channel 220, a light source connected to the channel 212, and an aspirator connectable to the channel 220 for aspirating fumes generated by coagulation of tissues. In the exemplary embodiment, the system and method are employed for Doppler or ultrasound guided thermal treatment of hemorrhoidal arteries. The anus-scope 200 is inserted into the anal canal above the hemorrhoidal cushings. The monitoring probe, such as the Doppler and/or ultrasound probe, is inserted into the channel 220 to identify the branches of the superior hemorrhoidal arteries. The scope 200 if rotated by, for example, manipulating the handle 210, until the monitoring probe identifies the respective hemorrhoidal artery. Then, the scope 200 is held in place, the monitoring probe is removed from the channel 220, and the energy application device is inserted into the channel 220 with the working end of the device positioned up to the rectal mucosa, or otherwise positioned closely adjacent to or in contact with the respective hemorrhoidal artery. The energy is emitted for a predetermined period of time sufficient to thermally damage the tissue and, in turn, significantly reduce and/or terminate the flow of blood through the respective hemorrhoidal artery.

In some such embodiments, the system and method are used for laser treatment of the hemorrhoidal arteries. In one such embodiment, the laser is a 980 nm laser, the fiber optic line is a 1000 micron fiber, and the monitoring device is an ultrasound Doppler probe (about 2 mm in diameter and that operates at about 20 MHz). The emitting end of the fiber optic is positioned up to the rectal mucosa and pulsed laser energy is applied a plurality of times. In one embodiment, the laser energy is applied about four times to each artery, each time at about 18 joules for a total of about 72 joules, at about 14 watts.

After application of the laser energy to the respective artery, the energy application device may be removed and the monitoring device re-inserted into the channel 220 to monitor the reduction in blood flow and/or closure of the respective artery resulting from the thermal treatment. The procedure is repeated by rotating the anus-scope 200 to each of the respective submucosal branches of the superior hemorrhoidal arteries. A typical procedure involves thermally treating about three arterial branches.

In other embodiments of the invention, rather than employ a laser and a fiber optic line, an RF generator and RF probe are used instead. In these embodiments, the RF probe is placed in contact with the respective arterial branches to thermally damage the branches and, in turn, significantly reduce or terminate blood flow. As may be recognized by those of ordinary skill in the pertinent art based on the teachings herein, any of numerous different energy sources, energy application devices and monitoring devices, that are currently known, or that later become known, may be employed in the system and method of the present invention.

In all currently preferred embodiments, no anesthetic or analgesic procedure is necessary, the procedure is ambulatory, is performed within a matter of minutes (e.g., within the range of about 10 to about 30 minutes, and most preferably within less than about 15 or 20 minutes), and the patient is released shortly after performing the procedure. The thermal treatment, such as by laser coagulation or photocoagulation, causes a necrosis of mucosal and submucosal tissues and closure or substantial closure of hemorrhoidal arteries. In patients treated it has been found that the endoscopic necrotic aspect persists for about 7 to 14 days after the procedure, that retraction of the hemorrhoidal cushings is evident also within about 7 to 14 days after the procedure, and that the retraction may increase for an additional period of time up to about 90 days, resulting in complete or partial reduction of prolapse. Accordingly, one advantage of the currently preferred embodiments of the invention is that thermal coagulation of the branches of the superior hemorrhoidal arteries, such as by laser treatment, both closes the arteries and fixes the rectal mucosa and submucosa to the muscular layer, causing a retraction of the hemorrhoids and an impediment to their prolapse through the anal canal. Yet another advantage is that the procedure causes minimal pain or discomfort to the patient and the post operative recovery can be faster than with alternative approaches and with very few complications. Yet another advantage is that anesthesia or analgesia are not required, thereby significantly reducing complications, simplifying treatment, and making the procedure faster and safer.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims. For example, any of numerous different energy sources, energy application devices, and/or monitoring devices may be employed. For example, any of numerous different laser or other intensive radiation devices may be employed, such as diode lasers or super-luminescent diodes either coupled to optical fibers having output ends that can be positioned in the vicinity of the feeder arteries or otherwise as required, or that are mounted on supports that allow their positioning at the vessels as desired or otherwise required. Further, the anus-scope illustrated herein is only exemplary, and may take any of numerous different configurations that are currently known, or that later become known. Accordingly, the term anus-scope is used herein to mean any of numerous different devices that are currently known, or that later become known for performing one or more of the functions of the anus-scope disclosed herein, including introduction of a monitoring device into the anal canal and/or introduction of an energy application device into the anal canal. Accordingly, this detailed description of currently preferred embodiments is to be taken in an illustrative as opposed to a limiting sense.

What is claimed is:

1. A treatment system for treating hemorrhoidal arteries comprising:
an anus-scope defining at least one of a size and shape for passage into and out of the anal canal and defining a channel therethrough;
a monitoring device receivable within the channel for identifying the hemorrhoidal arteries and monitoring blood flow therein; and
an energy application device having a working end and receivable within the channel for positioning the working end at a respective hemorrhoidal artery and, in turn, applying energy to the hemorrhoidal artery to thermally damage the artery.

2. The system according to claim 1, further comprising an energy source coupled to the energy application device.

3. The system according to claim 2, wherein the energy source is one of a laser and an RF generator.

4. The system according to claim 3, wherein the energy application device is one of a fiber optic and an RF probe.

5. The system according to claim 4, wherein the fiber optic defines a diameter within the range of about 200 microns to 2000 microns.

6. The system according to claim 1, wherein the monitoring device is one of a Doppler device and an ultrasound device.

7. The system according to claim 6, wherein the Doppler device is selected from the group consisting of a Doppler, an echo-Doppler, and an echo-color-Doppler.

8. The system according to claim 6, wherein the Doppler device is received within the channel and internally identifies the branches of hemorrhoidal arteries.

9. The system of claim 1, further comprising an aspirating device connectable in fluid communication with the channel for aspirating fumes therefrom.

10. The system according to claim 1, further comprising a light source optically coupled to the anus-scope for at least one of illuminating an interior of the anus-scope and illuminating a treatment site.

11. The system according to claim 1, wherein the anus-scope includes a plurality of channels for receiving the energy application device and the monitoring device, respectively.

12. The system according to claim 1, wherein the anus-scope is disposable.

13. The system according to claim 1, further including:
a. a first channel for receiving an illumination device illuminating at least one of an interior of the anus-scope and a treatment site; and
b. a multi-use channel for receiving therein either the energy application device or the monitoring device.

14. A treatment system for treating hemorrhoidal arteries comprising:
first means for passage into and out of the anal canal and defining a channel therethrough;
second means receivable within the channel for locating a hemorrhoidal artery and monitoring blood flow therein; and
third means receivable within the channel for applying energy to and thermally damaging the hemorrhoidal artery and at least one of significantly reducing and preventing blood flow therein.

15. The system of claim 14, wherein the first means is an anus-scope, the second means is a monitoring device, and the third means is an energy application device.

16. The system according to claim 15, wherein the monitoring device is one of a Doppler device and an ultrasound device, and the energy application device is one of a fiber optic and an RF probe.

17. A method for treating hemorrhoidal arteries in and around anal and rectal regions, comprising the steps of:
i. introducing an anus-scope into an anal canal;
ii. introducing a monitoring device through the anus-scope and into the anal canal, and locating with the monitoring device a hemorrhoidal artery; and
iii. introducing an energy application device through the anus scope and into the anal canal, positioning a working end of the energy application device at a hemorrhoidal artery identified by the monitoring device, applying energy from the working end of the energy application device to the hemorrhoidal artery and, in turn, thermally damaging the hemorrhoidal artery and at least one of significantly reducing and preventing blood flow therein.

18. The method according to claim 17, further comprising the step of placing the working end of the energy application device in contact with or closely adjacent to the hemorrhoidal artery.

19. The method according to claim 17, further comprising the steps of locating and treating a first hemorrhoidal artery, rotating the anus-scope and locating and treating a second hemorrhoidal artery.

20. The method according to claim 17, further comprising the steps of introducing the monitoring device through the anus-scope, rotating the anus-scope within the anal canal until the monitoring device locates a respective hemorrhoidal artery, fixing the position of the anus-scope at the respective hemorrhoidal artery, removing the monitoring device from the anus-scope, introducing the energy application device through the anus-scope and positioning the working end of the anus-scope at the hemorrhoidal artery, applying energy from the working end of the energy application device to the hemorrhoidal artery, and repeating the foregoing steps for at least one other hemorrhoidal artery.

21. The method according to claim 20, further comprising the steps of removing the energy application device from the anus-scope after thermally treating a hemorrhoidal artery, re-introducing the monitoring device into the anus-scope and monitoring the blood flow in the treated hemorrhoidal artery.

22. The method of claim 17, wherein the step of introducing an energy application device includes introducing an intensive radiation device through the anus scope and into the anal canal, positioning an output end of the intensive radiation device at a hemorrhoidal artery located by the monitoring device, applying sufficient radiation from the output end of the intensive radiation device to the hemorrhoidal artery to thermally damage the hemorrhoidal artery and at least one of significantly reduce and prevent blood flow therein.

23. The method of claim 17, further comprising illuminating at least one of an interior of the anus-scope and a treatment field during steps ii and iii.

24. The method of claim 17, further comprising aspirating a treatment site through the anus-scope during or following step iii.

25. The method of claim 16, further comprising performing steps I through iii without anesthesia.
